(19) United States
(12) Patent Application Publication
(10) Pub. No.: US 2007/0031327 A1
(43) Pub. Date: Feb. 8, 2007

(54) Nanoradiopharmaceuticals and methods of use

(76) Inventors: David E. Luzzi, Wallingford, PA (US); Brian W. Smith, Philadelphia, PA (US)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 10/559,446

(22) PCT Filed: Jun. 3, 2004
(86) PCT No.: PCT/US04/17479

§ 371 Date: May 9, 2006

Related U.S. Application Data
(60) Provisional application No. 60/475,526, filed on Jun. 3, 2003.
(60) Provisional application No. 60/495,369, filed on Aug. 15, 2003.

(51) Int. Cl. A61K 51/00

(52) U.S. Cl. 424/1.11; 977/906

(57) ABSTRACT

The present invention is directed to novel compositions and the use thereof More particularly, the present invention relates to nanoradiophannaceuticals, radioactive nanoparticles, and methods of using them for a range of applications including diagnostic imaging and the treatment of disease.

### CROSS-REFERENCE TO RELATED APPLICATIONS

This application claims priority to U.S. Provisional Application Ser. No. 60/495,369 filed Aug. 15, 2003 and U.S. Provisional Application Ser. No. 60/475,526 filed Jun. 3, 2003, each of which is incorporated herein by reference in its entirety.

### FIELD

The present invention is directed to novel compositions and the use thereof. More particularly, the present invention relates to nanoradiopharnaceuticals, radioactive nanoparticles, and methods of using them for a range of applications including diagnostic imaging and the treatment of disease.

### BACKGROUND

Nanoparticles have been used in many biological applications since the discovery that particles of such small size could be readily employed to deliver drugs, genes, contrast agents and vaccines into biological targets of interest such as cells and tissues. Oyewumi et al., International Journal of Pharmaceutics, 2003, 251: 85-97. Liposomes and polymersomes, for example, useful for their ability to encapsulate biomolecules, have been studied as possible intravenous drug delivery vehicles. Harrington et al., Joural of Pharmacy and Pharmacology 2002, 54: 1573-1600, Discher et al., Science 1999, 284: 1143-1146. Although such vesicles at about 100 nanometers are too large to extravasate through normal blood vessels, they are able to permeate the fenestrated endothelium of tumor neovasculature and the fenestrated or discontinuous endothelium of the liver, spleen, marrow sinusoid, and lung.

The delivery of cytotoxic agents, such as radioisotopes, to a subject is known as an effective way to treat certain diseases, including neoplastic diseases. The recent development of antibody-based targeting strategies has provided a method to deliver radioisotopes to tumors with decreased normal tissue toxicity. Treatment with antibody-conjugated radioisotopes, or radioimmunotherapy (RAIT), has demonstrated significant efficacy in the treatment of hematological malignancies. However, RAIT has proven to be relatively ineffective in the treatment of solid malignancies. Additionally, complications arise from the non-specific accumulation of radiation in normal tissues.

Alternative methods of delivering radionuclides to selected cells and tissue are needed. Known methods of delivering bioactive agents, however, suffer from many limitations that make them unsuitable for the delivery of radionuclides. For example, the time required for localization of liposomes and other particles of similar size to a delivery site make them unsuitable for the delivery of rapidly decaying radionuclides. Radioactive colloids and microspheres incorporating gold-198, yttrium-90, phosphorous-32, technicium-99, rhenium-186, and rhenium-188 have been prepared, however attempts to inject such radiocolloids intravenously have resulted in gross accumulation in the liver and lung due to RES phagocytosis and capillary obstruction, respectively. Jeong et al., Applied Radiation and Isotopes 2000, 52: 851-855. Such procedures are, thus, inappropriate for many therapeutic uses.

Accordingly, a need exists for novel compositions capable of effectively delivering radiation to specific sites in a subject. The present invention is directed to this, as well as other, important ends.

### SUMMARY

This invention provides, inter alia, methods for the preparation of nanoradiotherapeutics, nanoradiodiagnostics, nanoradiopharmaceuticals, nanoradiopharmacuetical compositions and nanoradioparticles for use therein. The nanoparticles are prepared in an aqueous medium through reduction of a radionuclide—containing moiety by a reducing agent. The conditions in the aqueous medium are such that nanoparticles are formed having mean diameters of from about 1 to about 25 nanometers. Ligand can then be associated with the nanoradioparticles thus formed, which ligand is specific for a biological target. The nanoradioparticles thus formed and their respective compositions can be prepared very rapidly and in such a way that radionuclides having relatively short half lives can be employed as the radioactive species. The nanoparticles and compositions can then be delivered to a biological system, organism, patient, animal, tissue, organ or cell preparation where the particles rapidly associate with the biological target.

The present methodology, which permits rapid synthesis of the nanoradioparticles, facilitates the delivery of relatively large doses of radiation to the locus of the biological target with relatively low amounts of non-specific radiation delivery elsewhere in the organism, tissue, organ, and the like. Since the half lives of the radionuclides which can be used is short, rapid loss of total radiation levels ensues. In short, the present invention delivers high doses of radiation to the locus of a biological target in a very specific way, with only very low amounts of non-specific irradiation together with an overall relatively low total irradiation.

In accordance with preferred embodiments, radionuclides are employed having a half life of from about 1 to about 100 hours, with half lives of from about 2 to about 50 hours being preferred and from about 3 to about 10 hours being more preferred. Mean particle diameters of from about 1 to about 15 nanometers are preferred with diameters of from about 2 to about 5 nanometers being more preferred. Synthesis time preferably is less than about 24 hours with shorter times, such as less than about 8 or even about 5 or about 2 hours being more preferred. The selection of the radionuclide can be matched to the anticipated synthesis time to ensure a preselected radiation dosage to the eventual biological target.

Exemplary radionuclides can include, for example, Rhenium 186, Rhenium 188, Copper 64, Copper 67, Gold 198, Gold 199, Silver 111, Rhodium 105, Palladium 109, Iridium 194, Technetium 99, Technetium 99 m, Technetium 94, and mixtures thereof. Rhenium moieties are preferred for some embodiments. Following reduction, the metal moieties can be conveniently provided as metals or metal containing compounds such as metal oxides or metal sulfides. Exemplary radionuclide-containing moieties of the present invention are those that are easily amenable to reduction, i.e., they have a positive reduction potential relative to the hydrogen half cell.

Unlike many nanoparticle synthetic schemes, the present invention prepares nanoradioparticles in aqueous medium. This preparation facilitates subsequent association of ligand with the nanoparticles and permits the rapidity of the preparation. This is accomplished by the aqueous reduction of a radionuclide, especially an oxy—anion of a radionuclide, by a reducing agent, such as metal hydride, especially a borohydride. The synthesis medium is preferably acidic, with a pH of from about 4 to about 7, although mildly basic pHs can be employed. Thus a pH range of from about 6 to about 9 can also be employed. PH is one property which can be controlled to tailor the physical properties of nanoradioparticles in accordance with this invention. Thus, size, and other properties can be varied through judicious selection of pH, reducing agent, adjuvants, synthesis procedures and other factors.

For use herein, the term “aqueous medium” refers to a medium comprising water wherein water is preferably the dissolving medium but need not be. In some embodiments, for example, other polar protic solvents that are compatible with administration to the body can be used as the dissolving medium, e.g., ethanol. When water is the dissolving medium, the medium will typically comprise at least about 80% water, more preferably at least about 90% or 95% by weight water. In preferred embodiments, the aqueous medium will have less than about 20% by weight of an organic solvent that is incompatible with administration to the body (e.g., a toxic-substance that must be removed from the medium before administration), preferably less than about 10% by weight organic solvent, even more preferably less than about 5% or 2% by weight organic solvent. In some preferred embodiments, the nanoradiopharmaceuticals are prepared in the absence of an organic solvent that is incompatible with administration to the body.

The ligand species which are preferred for association with the nanoradioparticles of this invention are those which are capable of causing the particles to come physically close to and to remain in the vicinity or locus of a biological target. Association of ligand with nanoparticles can occur in any effective way, such as covalent bonding, elaboration of a charge transfer complex, or association in any other way. Preferred ligands are immunologically active and participate in an antigen—antibody type of interaction with the chosen biological target. Monoclonal antibodies are preferred ligand species, especially a single chain scfv molecule. Ligand which localize nanoparticles through non-immunological interactions are also provided herein. Thus an atom or molecule can be associated with nanoparticles which cause them to localize at the biological target. One example of this is Iodine, which associates with the nanoradioparticles through a charge transfer complex and localizes the particles in the thyroid. Other ligands with similar capabilities can also be used.

The nanoradioparticles of the present invention can be in compositions further including one or more stabilizing or performance enhancing materials. Exemplary among these are polymers which keep the particles in effective suspension or which interfere with agglomeration or other undesired association. Polyoxyalkylene polyol species, such as polyethylene glycols and the like are preferred for some embodiments. Biopolymers such a collagen and the like can also find utility herein.

In accordance with some embodiments, constructs comprising nanoradioparticles can be prepared by placing or growing a coating on the nanoradioparticle. Accordingly, methods of preparing nanoradiopharmaceuticals can include the steps of reducing a radionuclide-containing moiety in aqueous medium with a reducing agent under conditions selected to form particles having a mean diameter of from 1 to about 25 nanometers; and growing a surface coating on the particles. In one embodiment, for example, a metal containing moiety is added to the medium in order to form the surface coating. A second reducing agent that is capable of reducing the metal containing moiety can be added to the medium. The present methods can further comprise the step of associating with the coated particles ligand(s) specific for a biological target. Surface coatings can be for example, inorganic coatings such as for example, carbon nanotubes and graphitic cages, metal coatings, such as, for example, gold or silver, or oxide coatings. The surface coating is preferably an inorganic, metal, or oxide surface coating (e.g., silicon oxide or titanium oxide).

Methods of growing surface coatings on nanoparticles are known although the performance of this procedure in the context of the present invention has not been known heretofore, see, for example, U.S. Pat. No. 6,544,463, Cao et al., J. Am. Chem. Soc., 2001, 123(32):7691-92, and Li et al., J. Phys. Chem. B. 105, 2001, 11424-11431, each of which is incorporated herein by reference in its entirety.

It is preferred for some embodiments that the biological target for the nanoradioparticles be implicated in a disease state, especially in microbial infection, tumorigenesis or development. It is often desired to destroy the locus or vicinity of the target. In the case of solid tumors, the locus or vicinity of the target can be the tumor itself. In the case of microbial pathogen, it can be the pathogen or cells infected with the pathogen. Use of a ligand which binds specifically to a cell receptor is preferred in some embodiments. The cell receptor can be an antigen, especially one implicated in a disease state, especially a neoplastic disease or microbial infection. The ligand species can also bind to a cell surface marker, especially one implicated or associated with a disease state, such a tumor endothelial marker or another antigen. The marker need not, itself, be implicated in a disease state so long as its location causes the nanoradioparticles to localize near a site to be usefully irradiated.

The present invention also provides nanoradiopharmaceuticals (e.g., nanoradiotherapeutics or nanoradiodiagnostics) comprising an aqueous dispersion of nanoradioparticles prepared in aqueous medium through reduction of radionuclide moieties by a reducing agent. The particles have ligand specific for a biological target and mean diameters of from 1 to about 25 nm. The pharmaceuticals can also contain stabilizers and other beneficial adjuvants consistent with their overall function.

In accordance with some embodiments, radiation is provided to the locus of a biological target in amount of from about 500 to about 8000 cGY or from about 500 to about 5000 cGY, with amounts of from about 1000 to about 2000 or from about 4000 to about 5000 cGY being preferred for some uses. Rates of irradiation can also be controlled with the present invention and irradiation rates to the locus of a biological target of from about 500 to about 3000 cGY, preferably from about 20 to about 750 cGY per hour, more preferably from about 50 to about 500 cGY per hour, calculated as an average over the first three hours of irradiation, can be attained hereby. Other dosage rates and amounts can be attained through routine variation of nanoradioparticle identity and application in view of the target locus to be irradiated. In accordance with one embodiment, tumor angiogenesis can be interfered with through use of the nanoradioparticle compositions hereof. Other neoplastic diseases can similarly be treated hereby.

Accordingly, the invention also provides pharmaceutical compositions comprising a therapeutically effective amount of a nanoradiopharrnaceutical in a pharmaceutically acceptable carrier or diluent. Other pharmaceutically acceptable adjuvants, stabilizers, antibiotics and the like can also be included in such compositions.

The invention thus provides nanoparticulate compositions which can have uses in imaging, diagnostics, research, and in industrial processes. The invention also provides methods for irradiating a selected biological target while minimizing irradiation of non-selected cells or tissues. These methods include contacting the target of a biological subject, tissue, organ, cell preparation or the like suspected of containing the target with nanoradioparticles in accordance with this invention.

Detection of the nanoradioparticles of this invention when they become present at the locus of the biological target permits imaging with high specificity, speed and efficiency. Scanning of a patient, animal, organ, tissue cell preparation or the like after contacting the same with a composition of this invention can give rise to detailed, useful data concerning the target area. Tomography, radiography, digital enhancement and other techniques can attend this process. The detection can be of positrons, gamma rays, electron spin, magnetic resonance information or other information streams deriving from the nanoparticles. Diagnosis of disease or of body state can be performed in conjunction with the imaging aspects of this invention.

Attendant to imaging in accordance with this invention, non-invasive surgery can be performed. Such surgery, colloquially called “gamma knife” surgery, applies transdermal radiation to a patient relying upon internal imaging as set forth above. Focusing a plurality of radiation sources upon the locus of the interaction of nanoparticles and biological target permits concentration of the radiation upon the locus of the target.

The present invention provides novel radiopharmaceuticals for diagnostic applications (e.g., nanoradiodiagnostics) and for therapeutic applications (e.g., nanoradiotherapeutics). In particular, the present invention provides nanoradiopharmaceuticals comprising radioactive nanoparticles associated with ligand moieties and, optionally, stabilizing materials for in vivo and in vitro applications. The present invention also provides methods of using the novel radiopharmaceuticals for a wide variety of applications including, for example, irradiating a selected biological target, imaging a selected biological target and selectively destroying tissue. The invention provides methods of diagnosing the presence of a disease state and methods of treating a disease or condition. In one preferred embodiment, the invention provides methods of treating cancer and in particular, methods of treating solid tumors. In another preferred embodiment, the invention provides methods of treating microbial disease. Other applications are also part of this invention. Thus, for example, through the use of the imaging aspects hereof, improved methods of radioimmuno-guided surgery can be attained.

Synthesis of Radioactive Nanoparticles

The present invention provides radioactive nanoparticles and methods of using them. As used in the present invention, the term “nanoradiopharmaceutical” refers to a plurality of radioactive nanoparticles or nanoradioparticles that are associated with a ligand, preferably one which is specific for a biological target. The nanoradiopharmaceuticals of the present invention can also comprise stabilizing material, e.g. certain polymers, which improve the stability of compositions containing the nanoparticles. The nanoradiopharmaceuticals can be used for diagnostic, research and therapeutic uses and can find utility in other areas of science as well. The nanoradiopharmaceuticals of the present invention can be easily dispersed in an aqueous buffer solution for administration into the body.

Use of the term “nanoparticle” refers to a particle having a size measured on the nanometer scale. While for some purposes, the nanometer scale runs from 1 to 1000 nanometers, for the purposes of the present invention, a nanoparticle is one having a mean diameter of from about one to about 100 nanometers in size. The mean diameter is measured indirectly, such as via transmission electron microscopy, TEM, or by other techniques known to persons skilled in the art of nanoparticles. Preferably, a nanoparticle of the present invention is from about one to about twenty-five nanometers in diameter, more preferably from about one to about ten nanometers in diameter, and even more preferably from about one to about three nanometers in diameter. In some embodiments of the present invention a nanoparticle is about one, two, three, four, five, six, seven, eight, nine or ten nanometers in diameter. It is to be understood that such sizes are measured in the average and generally as a number average of the particles rather than as a weight or volume average. It will also be understood that no particle size measurement is entirely precise and that there will nearly always be a distribution of particle sizes in any sample or preparation. Thus, this measurement should be seen to be a practical one given the nature of the invention. This facilitates the use of TEM to determine particle size. The morphology of the nanoradioparticles of the present invention can also be determined using TEM.

The nanoradiopharmaceuticals of the present invention are synthesized from radionuclide moieties. A radionuclide moiety of the present invention is a compound comprising a radioactive metallic isotope, which compound is capable of being reduced in an aqueous medium by a reducing agent to form a metal or metal containing composition in nanoparticulate form. The formation of nanoparticles from metallic precursors through aqueous reduction is known, per se, although the importance of performing this procedure in the context of the present invention has not been known heretofore.

Metallic radionuclides are commonly used as labeling reagents for antibodies in therapeutic and diagnostic applications. For example, radionuclides such as ¹¹C, ¹³N, ¹⁵O, ¹⁸F, ³²P, ⁵¹Mn, ⁵²Fe, ^{52m}Mn, ⁵⁵Co, ⁶²Cu, ⁶⁴Cu, ⁶⁷Cu, ⁶⁷Ga, ⁶⁸Ga, ⁷²As, ⁷⁵Br, ⁷⁶Br, ^{82m}Rb, ⁸³Sr, ⁸⁶Y, ⁸⁹Zr, ⁹⁰y, ^{94m}Tc, ⁹⁴Tc, ⁹⁵Tc, ^{99m}Tc, ¹¹⁰In, ¹¹¹In, ¹²⁰I, ¹²³I, ¹²⁴I, ¹²⁵I, ¹³¹I, ¹⁵⁴⁻¹⁵⁸Gd, ¹⁷⁷Lu, ¹⁸⁶Re, and ¹⁸⁸Re have been used as labeling reagents for diagnostic imaging techniques. Radionuclides such as ⁵¹Cr, ⁵⁷Co, ⁵⁸Co, ⁵⁹Fe, ⁶⁷Cu, ⁶⁷Ga, ⁷⁵Se, ⁹⁷Ru, ^{99m}Tc, ¹¹¹In, ^{114m}In, ¹²³I, ¹²⁵I, ¹³¹I, ¹⁶⁹Yb, ¹⁹⁷Hg, and ²⁰¹Tl have been used labeling reagents for diagnostic imaging techniques using gamma-ray detection methods. Radionuclides such as ³²P, ³³P, ⁴⁷Sc, ⁵⁹Fe, ⁶²Cu, ⁶⁴Cu, ⁶⁷Cu, ⁶⁷Ga, ⁷⁵Se, ⁷⁷As, ⁸⁹Sr, ⁹⁰Y, ⁹⁹Mo, ¹⁰⁵Rh, ¹⁰³Pd, ¹⁵⁹Gd, ¹⁴⁰La, ¹⁶⁹Yb, ¹⁷⁵Yb, ¹⁶⁵Dy, ¹⁶⁶Dy, ¹⁰⁵Rh, ¹¹¹Ag, ¹⁹²Ir, ¹⁰⁹Pd, ¹¹¹Ag, ¹¹¹In, ¹²⁵I, ¹³¹I, ¹⁴²Pr, ¹⁴³Pr ¹⁴⁹Pm, ¹⁵³Sm, ¹⁶¹Tb, ¹⁶⁶Ho, ¹⁶⁶Dy, ¹⁶⁶Ho, ¹⁶⁹Er, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹³Bi, ¹⁰⁶Ru, ²¹²Pb, ²¹³Bi, ²²³Ra and ²²⁵Ac has been used in therapeutic applications, such as the targeting of a radiolabeled antibody to a cancer cell. Any radioactive species, such as those listed above, having a reasonable half life in the context of this invention which is also capable of aqueous medium reduction to nanoparticles of the correct size can be employed in the nanoradiodiagnostics or nanoradiotherapeutics invention so long as the overall goals of the invention are maintained. A nanoparticle of the present can comprise a collection of homogenous or heterogeneous radionuclides. For example a nanoparticle can contain only Re¹⁸⁶ or both Re¹⁸⁶ and Re¹⁸⁸. In a preferred embodiment, radiopharmaceuticals of the present invention comprise isotopes of At, I, Ru, Cu, As, Rh, Pd, Ir, Ag, Re, Au, Bi, Tc or mixtures thereof.

The choice of radionuclide for use in the present invention takes into account several of the physical and chemical properties possessed by the radionuclide including the type of radiation emitted by the radionuclide. Radionuclides of the present invention can be alpha, beta, gamma, positron or Auger electron emitters.

The choice of radionuclide also takes into account the energy emission spectrum and the half-life of the radionuclide. For example, the energy emission spectrum and half-life of a radionuclide can be used to calculate the intrinsic radiotherapeutic or radiodiagnostic potency of a radionuclide. A general review of several of the considerations to be taken into account when choosing an appropriate radionuclide can be found in O'Donoghue, J. A. Dosimetric principles of targeted radiotherapy; P. G. Abrams and A. R. Fritzberg (eds.), Radioimmunotherapy of Cancer. New York, N.Y.: Marcel Dekker, 2000; and Goldenberg, J Nucl Med 2002, 43: 693-713, the disclosures of which are incorporated by reference in their entireties and for all purposes.

For use in the diagnostic applications of the present invention, useful decay energies can be, for example, in the range of about 20 to about 4,000 keV, more preferably in the range of 25 to 4,000 keV, and even more preferably in the range of about 20 to about 1,000 keV, and still more preferably in the range of about 70 to about 700 keV. Total decay energies of useful positron-emitting radionuclides can be, for example, less than about 2,000 keV, more preferably under about 1,000 keV, and most preferably under about 700 keV. Decay energies of useful gamma-ray emitting radionuclides can be, for example, about 20 to about 2000 keV, more preferably about 60 to about 600 keV, and most preferably about 100 to about 300 keV.

For use in the therapeutic applications of the present invention, useful decay energies can be, for example, in the range of about 0.001 to about 6,000 keV, in the ranges about 0.001 to about 2 keV for an Auger emitter, about 100 to about 2,500 keV for a beta emitter, and about 4,000 to about 9,000 keV for an alpha emitter.

Radionuclides for use in the present invention have a half-life that is compatible with nanoparticle synthesis and the subsequent diagnostic, imaging, therapeutic, industrial or other use of the nanoradioparticle. Preferably, a nanoradiopharmaceutical is administered to a subject or used in in vitro applications before an undesirable amount of decay has occurred, thereby permitting a lower dose of the nanoradiopharmaceutical. Radionuclides that have relatively long half-lives can emit radioactivity long after the desired dose has been achieved, such that the radionuclide is selected to optimize dosage time and conditions so as to preferably deliver a selected radiation dosage to a biological target vicinity or locus on a predetermined time scale. Accordingly, a balance must be reached so that an effective therapeutic or diagnostic amount of radiation is reaching the desired location. A skilled practitioner will to be able to make a determination of which radionuclides have an appropriate half-life for use in the methods of the present invention given the particular circumstances at hand. Radionuclides with appropriate half-lives include, for example, those having the preferred isotopes, ¹⁸⁶Re, ¹⁸⁸Re, ⁶⁴Cu, ⁶⁷Cu, ¹⁹⁸Au, ¹⁹⁹Au, ⁷⁷As, ¹⁰⁵Rh, ¹⁰⁹Pd, ¹¹¹Ag, ¹⁹⁴Ir, and ^{99m}Tc. As guideline, a radioactive half-life of from about 1 to about 100 hours, or from about 2 about 50 hours, or from about 3 to about 15 hours is preferred for therapeutic or diagnostic use although research and industrial applications may suggest longer or shorter half lives.

Radionuclides suitable for use in the present invention include alpha, beta, gamma, positron or Auger electron emitters capable of quick reduction in aqueous medium to form metallic compounds from about 1 to about 25 nanometers in diameter, more preferably from about 1 to about 3 nanometers in diameter. One nanoparticle synthesized using the methods of the present invention and having a diameter of about 1 to 3 nanometers can comprise several, tens or hundreds of individual radionuclides. For example, a nanoradioparticle of about 2-3 nm in diameter can comprise from about 100 to about 500 individual radionuclides. In some embodiments at least about 5%, 10%, 20%, 30%, 40%, 50%, or 60% of the individual radionuclides are radioactive. Accordingly, in one embodiment of the present invention, a nanoradiopharmaceutical can comprise about 5, 10, 15, 20, 25, 30, or 300 radioactive radionuclides attached to one or more ligands. The average number of radioactive atoms per nanoparticle is preferably at least 2, more preferably at least about 5, and even more preferably at least about 6, 8, 10, 15, or 20.

There are at least two alternative methods for measuring the average number of radioactive atoms per particle (e.g., nanoparticle). In some situations, one method may work better than the other. In some of the claimed embodiments, the average number of radioactive atoms per particle in a nanoradiopharmaceutical is provided. In determining whether a particular average number of atoms per nanoparticle is present as required by certain of the claims hereof, the limitation is met if either measurement methodology produces the required figure.

In one of the methods for measuring the average number of radioactive atoms per particle, the concentration of nanoparticles in a sample (e.g., in a nanoradiopharmaceutical) is measured by optical absorption (i.e., using Beer's Law) and the radioactivity of the sample is measured using a dose calibrator. The measurement of the radioactivity provides the number of radioactive atoms in the sample. The number of nanoparticles in the sample is determined by multiplying the volume of the sample by the concentration. The number of radioactive atoms divided by the number of nanoparticles provides the average number of radioactive atoms per nanoparticle in the sample.

In an alternative method, the average size, volume, and composition of the nanoparticles in a sample is ascertained by conventional characterization methods (e.g., transmission electron microscopy). Density is determined from the composition and the mass of each particle is calculated by multiplying the density by the volume. Using the known stoichiometric ratio of the composition and the mass of each particle, the number of atoms per nanoparticle is calculated. The number of radioactive atoms in the starting material is used to obtain the average number of radioactive atoms per nanoparticle in the final product. For example, if there are 100 atoms per nanoparticle in the final product and in the starting material 5% of all atoms were radioactive, there will be on average 5 radioactive atoms per nanoparticle in the final product.

It is one object of the present invention to provide methods of preparing nanoradiopharmaceuticals. Accordingly, the present invention provides a method of preparing a nanoradiopharmaceutical comprising reducing a radionuclide-containing moiety in aqueous medium with a reducing agent under conditions selected to create radioactive nanoparticles in the form of metal compounds.

As used herein, the phrase “reducing agent” means a compound that reacts with a moiety in a relatively oxidized form, for example, a metallic radionuclide in a relatively high oxidation state. The reducing agent acts to lower its oxidation state by transferring electron(s) to the radionuclide. The resulting, reduced material, preferably a metal oxide, where the metal contains radioactive isotopic species, can attain the form of nanoparticles with controlled mean diameters. Suitable reducing agents are those that are capable of quickly reducing a radionuclide moiety in accordance with the present invention. Metal hydrides, especially borohydrides such as sodium borohydride are preferred. By controlling the rate of reduction of the radionuclide, the size of the nanoparticles can be controlled. Faster reduction rates result in smaller particles. In one aspect, the reduction rate can be controlled by hydrogen ion concentration, e.g., pH. Preferably the reduction reaction will go to completion in less than about twenty-four hours, preferably less than about eight hours, less than about five hours, less than about two hours, and even more advantageously, less than about one hour. In an exemplary embodiment, nanoparticle synthesis will be accomplished in from about 15 to about 300 minutes.

Suitable reducing agents for the synthesis of the radioactive nanoparticles of the present invention include, but are not limited to, stannous salts, dithionite or bisulfite salts, borohydride salts, and formamidinesulfinic acid, wherein the salts are of any pharmaceutically acceptable form. The amount of a reducing agent used will depend upon the amount of radionuclide to be reduced and can be determined by a skilled practitioner. Reducing agents are chosen dependent on the radionuclide to be reduced. The reducing agents are those that can be used in aqueous solutions. For example, for reduction of rhenium isotopes, a preferably reducing agent is a metal hydride appropriate for use in aqueous solution, e.g., borohydride. In one aspect, reducing agents that do not reduce carboxylic acids are selected for use. In some embodiments, a metal surface coating will be placed on the nanoradioparticle thus requiring a second reduction step. The reducing agent will be any reducing agent capable of reducing the metal containing moiety in order to provide the surface coating. In one embodiment, a preferred metal containing moiety is silver chloride and the preferred reducing agent is sodium borohydride.

In addition to a radionuclide-containing moiety and reducing agent, an acidic condition may be favorable and preferred for synthesis. In certain embodiments, the molar ratio of reactants in the synthesis reaction is about 1:1-5:15 (radionuclide-containing moiety to acid to reducing agent). In one aspect, the reactions are performed under continuous sonication to prevent agglomeration of the unprotected nanoparticles as they grow.

In a further embodiment, the present invention provides rhenium oxide nanoparticles and methods of preparing them. The reaction involves the addition of sodium borohydride to a mixture of sodium perrhenate and an acid, e.g., acetic acid or ascorbic acid. The relative amount of acid determines the rate of reduction, thereby regulating size control. For perrhenate to acetic acid to borohydride ratios of 1:1-5:15, the synthesis reaction goes to completion in less than about one hour. The reaction product is the mixed oxide ReO₂/Re₂O₃.

The synthesis reaction takes place in an aqueous medium. In one embodiment, the aqueous medium has a pH of from about 4 to about 7. In another embodiment, the aqueous medium has a pH of from about 6 to about 9.

In one embodiment of the present invention, a final titration step is performed after nanoparticle synthesis and preferably after capping or coating of the nanoparticles with a thiol containing molecule, e.g., thioglycolic acid. The final titration adjusts the pH to one that is neutral or slightly basic e.g., a pH of about 7 to about 8. In one aspect, the nanoradiopharmaceuticals are suspended in an aqueous medium that is at physiological pH, e.g., at a pH of about 7.4. A “suspension” or a “dispersion” as used herein refers to a mixture, preferably finely divided, of two or more phases (solid, liquid or gas), such as, for example, solid in liquid, which preferably can remain stable for extended periods of time.

As used herein, the term “stable” refers to compounds which possess stability sufficient to allow manufacture and which maintains the integrity of the compound for a sufficient period of time to be useful for the purposes detailed herein. Typically, such compounds are stable at a temperature of 40° C. or less, in the absence of moisture or other chemically reactive conditions, for at least one day.

Association of the Radioactive Nanoparticles with One or More Ligands

The present invention provides radioactive nanoparticles that are preferably associated with one or more targeting agents or stabilizing moieties. It is contemplated that a nanoradioparticle can be associated with targeting agents specific for different biological targets or for the same biological target. It is also contemplated that a targeting agent can be associated with nanoradioparticles comprising a collection of heterogeneous radionuclides.

The term “associating with” when referring to a ligand of the present application refers to the attachment or bonding of the ligand to a nanoparticle of the present invention. The type of attachment can be, for example, through a covalent bond or through a charge transfer complex. As used herein the term “covalent bond” refers to an intermolecular bond which involves the sharing of electrons in the bonding orbitals of two atoms. The term “charge transfer complex” refers to an electron-donor-electron-acceptor complex characterized by electronic transition(s) to an excited state in which there is a partial transfer of electronic charge from the donor to the accepting moiety.

In a preferred embodiment of the present invention, the radioactive nanoparticles are associated with one or more ligands via a metal thiolate bond. In these embodiments, the ligands are thiolated and introduced to the nanoparticles for binding. For example, a thiolating agent such as 2-iminothiolane can be used to convert the amine of an amine-terminated ligand such as, for example, polyethylene glycol, to a thiol. The thiolated ligand can then be reacted with the nanoparticle and attached via a metal-thiolate bond.

In some embodiments it is desired to attach a peptide such as, for example, an antibody to the nanoparticles of the present invention. In such embodiments, the peptide can be synthesized to have distal cysteine residues, each of which has a free thiol group. The free thiol group can attach directly to the nanoparticle. Alternatively, the primary amines in a peptide can be first thiolated with a thiolating agent, e.g., 2-iminothiolane, and then attached to the nanoparticle via a metal-thiolate bond.

In certain embodiments, a thiol containing molecule such as thioglycolic acid, (e.g., mercaptoacetic acid), dimercaptosuccinic acid, or dihydrolipoic acid can be used to coat the nanoradioparticles prior to attachment of a targeting ligand. For example, following synthesis of the radioactive nanoparticles of the present invention, the nanoparticles can be reacted with mercaptoacetic acid to form mercaptoacetic acid-capped nanoradioparticles. The mercaptoacetic acid-capped nanoradioparticles can react with one or more targeting ligands.

In some embodiments, the targeting ligands can be attached to the nanoparticles via free carboxyl groups on the capping or stabilizing ligands. Following synthesis of the radioactive nanoparticles of the present invention, the nanoparticles can be reacted with mercaptoacetic acid to form mercaptoacetic acid-coated nanoradioparticle. The mercaptoacetic acid-coated nanoradioparticle coats the nanoparticles' surfaces with carboxyl groups. The carboxyl groups can then be used to bind targeting ligands of the present invention using known methods, e.g., coupled to the carboxyl groups using a carbodimide coupling agent, such as EDAC.

After attachment of the ligands to the nanoradioparticles, the functionalized nanoradioparticles can be purified by any of a number of techniques well known to those skilled in the art such as, for example, column chromatography or dialysis. Column chromatography includes the use of, for example, desalting columns, ion exhange columns, and affinity columns. For example, in one embodiment, after association of ligands to the nanoradioparticles, the aqueous solution containing the targeted nanoradioparticles will be eluted through an affinity column to remove all radioisotopes that have not been targeted as well as other undesirable by-products of the reaction. The isolated functionalized nanoparticles can thus be redispersed in a different aqueous solution, e.g., saline, for subsequent use.

The ligands of the present invention can be, for example, targeting agents or stabilizing materials. A “stabilizing material” or a “stabilizing compound” refers to any material which can improve the stability of compositions of the present invention, including, for example, mixtures, suspensions, emulsions, dispersions, vesicles, or the like. The improved stability involves, for example, the maintenance of a relatively balanced condition, and can be exemplified, for example, by increased resistance of the composition against destruction, decomposition, agglomeration, degradation, and the like. Exemplary stabilizing compounds which can be employed in the methods and compositions of the present invention include lipids, proteins and polymers, e.g., hydrophilic polymers. The polymer can be synthetic, naturally-occurring or semisynthetic, Exemplary water-soluble polymers that can used in the methods of the present invention, include poly(lactic-co-glycolic acid) (PLGA), poly(L-lysine)-g-poly(ethylene glycol) (PLL-g-PEG), poly(ethylene oxide) (PEO), and polyethylene glycol.

The present methods do not necessarily require a step of isolating intermediates after radionuclide reduction and before association of the nanoparticles with ligand. Accordingly radioactive nanoparticle synthesis and association of the nanoparticle with ligand can be carried out as a one pot synthesis. In some particularly preferred embodiments, the medium that is used to reduce the radionuclide is the same medium that is used to associate the nanoparticles to a targeting ligand. It is understood however that the medium can undergo changes, e.g., pH changes, or can have additional components added to it before ligand association. By the term “same medium” or “in said medium,” it is meant that the radioactive nanoparticles are not isolated or separated from the medium and redispersed in a new medium before ligand association. In some embodiments, however, the present invention does include the step of isolating intermediates after radionuclide reduction and before association of the nanoparticles with ligand. In such embodiments, the reducing step and association step can take place in different mediums.

Preferred methods of the present invention are ideally suited for use with nuclear pharmacies and kit based preparation methods of preparing nanoradiopharmaceuticals. In accordance with the present invention, kits are provided comprising non-radioactive ingredients (e.g., reducing agent, buffering agent, targeting ligand and/or stabilizing ligand, i.e., capping agent)) that can be used in the methods of the present invention in combination with the radionuclide-containing moiety to prepare the nanoradiopharmaceuticals of the present invention. The radionuclide-containing moieties of the present invention need not be activated (e.g, in an accelerator such as a cyclotron) after reduction and/or association with ligand because the reduction step is performed using a starting material that is already radioactive, i.e., the radionuclide-containing moiety. Preferably, the starting material is easily available from a nuclear pharmacy. In an exemplary embodiment, the radionuclide-containing moiety is perrhenate ion.

Targeting Ligands

The radioactive nanoparticles of the present invention can be associated with one or more targeting ligands to create the nanoradiopharmaceuticals of the present invention. The ligands can be specific for the same biological target or alternatively, for a different one. For example, the ligands can be specific for one or more different epitopes on a cell surface.

The phrases a “targeting ligand” or “targeting agent or “ligand specific for a biological target” are used interchangeably and refer to any material or substance which can promote targeting of a selected biological target including tissues, organs, cell, or collections of cells. In some embodiments, the selected biological target will be an invading pathogen, e.g., bacteria, virus, or parasite, and a ligand specific for the biological target will promote targeting of the pathogen and/or the site of infection within the body. For use in the present invention, a “ligand specific for a biological target” can be cross-specific meaning that it can be relatively specific for a class of biological targets. Accordingly, in some embodiments, the ligand will be relatively specific for a class of pathogens or cells infected by a class of pathogen.

As used herein, “tissue” refers generally to specialized cells which can perform a particular function. It should be understood that the term “tissue,” as used herein, can refer to an individual cell or a plurality or aggregate of cells, for example, membranes or organs. The term “tissue” also includes reference to an abnormal cell or a plurality of abnormal cells. Exemplary tissues include, for example, myocardial tissue (also referred to as heart tissue or myocardium), including myocardial cells and cardiomyocites, membranous tissues, including endothelium and epithelium, laminae, connective tissue, including interstitial tissue, and tumors, including, for example, solid tumors.

The targeting ligand can be synthetic, semi-synthetic, or naturally-occurring. Exemplary targeting ligands for use in the present invention include, but are not limited to proteins, including antibodies, glycoproteins and lectins, peptides, polypeptides, saccharides, including mono- and polysaccharides, vitamins, steroids, steroid analogs, hormones, cofactors, bioactive agents, and genetic material, including nucleosides, nucleotides and polynucleotides.

In some embodiments, the targeting agents specifically target receptors on or near selected biological targets. The term “receptor” as used herein refers to a molecular structure within a cell or on the surface of the cell which is generally characterized by the selective binding of a specific substance. Exemplary receptors include, for example, cell-surface receptors for peptide hormones, neurotransmitters, antigens, complement fragments, and immunoglobulins, cytoplasmic receptors for steroid hormones and receptors on invading pathogens. Receptors can be, for example, membrane bound, cytosolic or nuclear; monomeric (e.g., thyroid stimulating hormone receptor, beta-adrenergic receptor) or multimeric (e.g., PDGF receptor, growth hormone receptor, IL-3 receptor, GM-CSF receptor, G-CSF receptor, erythropoietin receptor and IL-6 receptor). In some embodiments, the targeting agents specifically target proteins on or near selected biological targets

The phrase “specifically bind(s)” or “bind(s) specifically” or “specifically targets” when referring to a targeting ligand refers to a ligand which has intermediate or high binding affinity, exclusively or predominately, to a particular biological target. The phrase “specifically binds to” refers to a binding reaction which is determinative of the presence of the biological target in the presence of a heterogeneous population of proteins and other biologics. Thus, under designated assay conditions, the specified binding moieties bind preferentially to a particular biological target and do not bind in a significant amount to other components present in a test sample. Specific binding to a biological target under such conditions can require a binding moiety that is selected for its specificity for a particular target antigen. A variety of assay formats can be used to select ligands that are specifically reactive with a particular biological target. Typically, a specific or selective reaction will be at least twice background signal or noise and more typically more than 10 times background. Specific binding between a ligand and its biological target typically can mean, for example, a binding affinity or an equilibrium association constant (Kₐ) of at least about 10³ M⁻¹, and preferably 10⁵, 10⁶, 10⁷, 10⁸, 10⁹ or 10¹⁰ M⁻¹. The term “Kₐ”, as used herein, is intended to refer to the equilibrium association constant of a particular ligand-receptor interaction, e.g., antibody-antigen interaction. This constant has units of 1/M. The term “K_{d}”, as used herein, is intended to refer to kinetic dissociation constant of a particular ligand-receptor interaction. This constant has units of M. “Particular ligand-receptor interactions” refers to the experimental conditions under which the equilibrium and kinetic constants are measured.

The specificity of the targeting ligand to a biological target will, in some embodiments, be independent of immunological interactions. For example, folic acid can be used as a targeting ligand to preferentially target cancer cells which overexpress the folate receptor. “Non-immunogenic in a human” means that upon contacting the ligand of interest in a physiologically acceptable carrier and in a therapeutically effective amount with the appropriate tissue of a human, no state of sensitivity or resistance to the ligand of interest is demonstrable upon the second administration of the ligand of interest after an appropriate latent period (e.g., 8 to 14 days).

Alternatively, in other embodiments of the present invention, the specificity of the ligand to the biological target will depend upon immunological interactions. For example, the ligand will be an immunologically active moiety, e.g., an antibody, and will bind to a region on a cell surface to which immunologically active moieties bind, e.g., an antigenic epitope. With respect to antibodies, the term, “immunologically specific” refers to antibodies that bind to one or more epitopes of a protein of interest, but which do not substantially recognize and bind other molecules in a sample containing a mixed population of antigenic biological molecules.

As used herein, an “antibody” refers to a protein consisting of one or more polypeptide substantially or partially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively. Antibodies exist as intact immunoglobulins or as a number of well characterized fragments produced by digestion with various peptidases. Thus, for example, pepsin digests an antibody below the disulfide linkages in the hinge region to produce F(ab)′₂, a dimer of Fab which itself is a light chain joined to VH—CH1 by a disulfide bond. The F(ab)′₂ can be reduced under mild conditions to break the disulfide linkage in the hinge region thereby converting the (Fab′)₂ dimer into an Fab′ monomer. The Fab′ monomer is essentially an Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments can be synthesized de novo either chemically or by utilizing recombinant DNA methodology. Thus, the term antibody, as used herein also includes antibody fragments either produced by the modification of whole antibodies or synthesized de novo using recombinant DNA methodologies. The term “antibody” is used in the broadest sense and specifically covers monoclonal and polyclonal antibodies, antibody compositions with polyepitopic specificity, chimeric antibodies, humanized antibodies, bispecific antibodies, diabodies, triabodies, tetrabodies, and single-chain molecules, as well as antibody fragments (e.g., Fab, F(ab′)₂, and Fv), so long as they exhibit the desired biological activity.

Methods of producing polyclonal and monoclonal antibodies that react specifically with biological targets are known to those of skill in the art and are thus not described herein in great detail. (see, e.g., Coligan, 1991, Current Protocols in Immunology; Harlow & Lane, supra; Goding, Monoclonal Antibodies: Principles and Practice (2d ed.); and Kohler & Milstein, Nature, 1986, 256:495-497). Such techniques include, for example, antibody preparation by selection of antibodies from libraries of recombinant antibodies in phage or similar vectors, as well as preparation of polyclonal and monoclonal antibodies by immunizing rabbits or mice (see, e.g., Huse et al., Science, 1989, 246:1275-1281; Ward et al., Nature, 1989, 341:544-546.

Antibodies or antibody fragments can be isolated from antibody phage libraries generated using the techniques described in McCafferty et al., Nature, 1990, 348:552-554. Clackson et al., Nature, 1991, 352:624-628 and Marks et al., J. Mol. Biol., 1991, 222:581-597. Subsequent publications describe the production of high affinity (nM range) human antibodies by chain shuffling (Mark et al., Bio/Technology, 1992, 10:779-783), as well as combinatorial infection and in vivo recombination as a strategy for constructing very large phage libraries (Waterhouse et al., Nuc. Acids. Res., 1993, 21:2265-2266). Thus, these techniques are viable alternatives to traditional monoclonal antibody hybridoma techniques for isolation of monoclonal antibodies.

Chimeric or hybrid antibodies also can be prepared in vitro using known methods in synthetic protein chemistry, including those involving crosslinking agents. For example, immunotoxins can be constructed using a disulfide-exchange reaction or by forming a thioether bond. Examples of suitable reagents for this purpose include iminothiolane and methyl-4-mercaptobutyrimidate. Methods for humanizing non-human antibodies are also well known in the art (Carter et al., Proc. Natl. Acad. Sci. USA, 1992, 89:4285; Presta et al., J. Immnol., 1993, 151:2623).

Bispecific antibodies (BsAbs) are antibodies that have binding specificities for at least two different antigens. BsAbs can be used as tumor targeting or imaging agents. Such antibodies can be derived from full length antibodies or antibody fragments (e.g. F(ab′)2 bispecific antibodies). Methods for making bispecific antibodies are known in the art (Millstein et al., Nature, 1983, 305:537-539, WO 94/04690 published Mar. 3, 1994, Suresh et al., Methods in Enzymology, 1986, 121:210)

Techniques for generating bispecific antibodies from antibody fragments have also been described in the literature. According to these techniques, Fab′-SH fragments can be recovered from E. coli, which can be chemically coupled to form bivalent antibodies. Shalaby et al., J. Exp. Med., 1992, 175:217-225 describe the production of a fully humanized BsAb F(ab′)₂ molecule. Each Fab′ fragment was separately secreted from E. coli and subjected to directed chemical coupling in vitro to form the BsAb. The BsAb thus formed was able to bind to cells overexpressing the HER2 receptor and normal human T cells, as well as trigger the lytic activity of human cytotoxic lymphocytes against human breast tumor targets. See also Rodriguez et al., Int. J. Cancers, (Suppl.) 1992, 7:45-50.

Various techniques for making and isolating bivalent antibody fragments directly from recombinant cell culture have also been described. For example, bivalent heterodimers have been produced using leucine zippers. Kostelny et al., J. Immunol., 1992, 148(5): 1547-1553. The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. The “diabody” technology described by Hollinger et al., Proc. Natl. Acad. Sci. USA, 1993, 90:6444-6448 has provided an alternative mechanism for making BsAb fragments. The fragments comprise a heavy-chain variable domain (VH) connected to a light-chain variable domain (VL) by a linker which is too short to allow pairing between the two domains on the same chain. Accordingly, the VH and VL domains of one fragment are forced to pair with the complementary VH and VL domains of another fragment, thereby forming two antigen-binding sites. Another strategy for making BsAb fragments by the use of single-chain Fv dimers has also been reported. See Gruber et al., J. Immunol., 1994, 152:5368.

The use of targeted nanoradiopharmaceuticals offers many opportunities for the treatment and/or diagnosis of various conditions and diseases. For example, a nanoradioparticle of the present invention can be associated with one or more antibodies that specifically recognize a tumor-specific parenchymal or vascular antigen. In one aspect, a nanoradioparticle of the present invention can be associated with one or more antibodies that specifically recognize a target antigen located on or within a tumor. The nanoradiopharmaceutical will localize to the tumor site to obtain diagnostic or therapeutic benefits. A nanoradioparticle of the present invention can be associated with one or more antibodies that specifically recognize a site of infection. In one aspect, a nanoradioparticle of the present invention can be associated with one or more antibodies that specifically recognize a target antigen located, for example, on or within a pathogen (e.g., virus, bacteria, or parasite) invading the body or on a cell infected with a pathogen. The nanoradiopharmaceutical will localize to the site to obtain diagnostic or therapeutic benefits.

It is known to use radiolabeled antibodies for radioimmunodetection and radioimmunotherapy techniques and in particular for cancer therapy. Certain combinations of antibodies and radiolabels have met with success in various different applications, Accordingly, it is one object of the present invention to synthesize radiopharmaceuticals comprising the same combinations of radionuclide and antibody known in the art. The radionuclide will be one that is amenable to nanoparticle synthesis. In this manner, tumors previously unresponsive or minimally responsive to radioimmunotherapy can be targeted and will absorb a greater amount radiation than possible with radiolabeled antibody therapy. The forced localization of multiple radionuclides all at the same time to a biological target provides for a high dose rate to the target while the total dose of radiation to the whole body remains low.

“Tumor cells” or “tumor” refers to an aggregate of abnormal cells and/or tissue which can be associated with (e.g., implicated in) diseased states that are characterized by uncontrolled cell proliferation. The disease states can involve a variety of cell types, including, for example, endothelial, epithelial and myocardial cells. Included among the disease states are cancers.

Tumor-associated antigens that can be targeted using the methods of the present invention include any antigen over-expressed on tumors such as, for example, A3, A33, BrE3, CD1, CD1a, CD3, CD5, CD15, CD19, CD20, CD21, CD22, CD23, CD30, CD37, CD45, CD74, CD79a, HLA-DR10, CEA, CSAp, EGFR, EGP-1, EGP-2, Ep-CAM, Ba 733, HER2/neu, KC4, KS-1, KS1-4, Le-Y, MAGE, MUC1, MUC2, MUC3, MUC4, PAM-4, PSA, PSMA, RS5, S100, TAG-72, tenascin, Tn antigen, Thomson-Friedenreich antigens, tumor necrosis antigens, VEGF, 17-1A, an angiogenesis marker, a cytokine, an immunomodulator, an oncogene marker and an oncogene product.

Tumor-associated markers have been categorized by Herberman (see, e.g., Immunodiagnosis of Cancer, in The Clinical Biochemistry of Cancer, Fleisher ed., American Association of Clinical Chemists, 1979, the disclosure of which is incorporated by reference in its entireties and for all purposes) in a number of categories including oncofetal antigens, placental antigens, oncogenic or tumor virus associated antigens, tissue associated antigens, organ associated antigens, ectopic hormones and normal antigens or variants thereof. Occasionally, a sub-unit of a tumor-associated marker is advantageously used to raise antibodies having higher tumor-specificity, e.g., the beta-subunit of human chorionic gonadotropin (HCG) or the gamma region of carcinoembryonic antigen (CEA), which stimulate the production of antibodies having a greatly reduced cross-reactivity to non-tumor substances as disclosed in U.S. Pat. Nos. 4,361,644 and 4,444,744, each of which is incorporated herein by reference in its entirety. Markers of tumor vasculature (e.g., VEGF), of tumor necrosis, of membrane receptors (e.g., folate receptor, EGFR), of transmembrane antigens (e.g., PSMA), and of oncogene products can also serve as suitable tumor-associated targets for antibodies or antibody fragments. Markers of normal cell constituents which are overexpressed on tumor cells, such as B-cell complex antigens, as well as cytokines expressed by certain tumor cells (e.g., IL-2 receptor in T-cell malignancies) are also suitable targets for the antibodies and antibody fragments of this invention.

Antibodies specific for tumor associated markers are well known and include, for example, the BrE3 antibody, (Couto et al., Cancer Res. 55:5973s-5977s (1995)); the EGP-2 antibodies (Staib et al., Int. J. Cancer 2001, 92:79-87; Schwartzberg et al., Crit. Rev. Oncol. Hematol. 2001, 40:17-24); the KS-1 antibody (Koda et al., Anticancer Res. 2001, 21:621-627); the A33 antibody (Ritter et al., Cancer Res. 2001, 61:6854-6859); the Le(y) antibody B3 (Di Carlo et al., Oncol. Rep. 2001, 8:387-392); and the A3 antibody (Tordsson et al., Int. J. Cancer 2000, 87:559-568).

Also of use are antibodies against markers or products of oncogenes, or antibodies against angiogenesis factors, such as VEGF. VEGF antibodies are described in U.S. Pat. Nos. 6,342,221, 5,965,132 and 6,004,554, the disclosures of which are incorporated by reference in their entirety and for all purposes. Antibodies against certain immune response modulators, such as antibodies to CD40, are described in Todryk et al., J. Immunol. Meth. 248:139-147 (2001) and Turner et al., J. Immunol. 2001, 166:89-94, the disclosures of which are incorporated herein by reference in their entirety and for all purposes. Other antibodies suitable for combination therapy include, for example, anti-necrosis antibodies as described in, for example, U.S. Pat. Nos. 5,019,368; 5,882,626; and 6,017,514, the disclosures of which are incorporated by reference in their entirety and for all purposes.

The compositions of the present invention are useful for treating solid tumors. Accordingly, the radioactive nanoparticles of the present invention can be attached to antibodies that specifically recognize a target antigen located on or within a solid tumor. Solid tumor-associated antigens that can be targeted using the methods of the present invention include, for example, any antigen over-expressed on solid tumors including, but not limited to, the tenascin antigen which is overexpressed in glial tumors; the EGFR antigen which is everexpressed in glial tumors; the E2 antigen which is overexpressed in leptomeningeal cancer; the MUC1 antigen which is overexpressed in ovarian carcinoma, breast cancer, and bladder cancer; glycoprotein which is overexpressed in renal cell carcinoma and ovarian carcinoma; the TAG-72 antigen which is overexpressed in colorectal cancer, ovarian carcinoma, prostate cancer, and breast cancer; the CEA antigen which is overexpressed in colorectal cancer, small cell lung cancer, and medullary thyroid cancer; the A33 antigen which is overexpressed in colorectal cancer; a pancarcinoma antigen which is overexpressed in colorectal cancer; the L6 antigen which is overexpressed in breast cancer; the TF antigen which is overexpressed in breast cancer, the PSMA antigen which is overexpressed in prostate cance; and ferritin which is overexpressed in hepatocellular carcinoma.

Antibodies specific for solid tumor associated markers are well known and include, for example, the BC4 antibody (Paganelli G, et al. Eur J Nucl Med. 1999; 26:348-357); the 816C antibody (Akabani G, et al. Int J Radiat Oncol Biol Phys. 2000; 46:947-958); the 425 antibody (Kalafonos, H P, et al. J Nucl Med. 1989; 30:1636-1645); the 3F8 antibody (Kramer K, et al. Med Pediatr Oncol. 2000; 35:716-718); the HMFG1 antibody (Epenetos A A, et al. Cancer Biother Radiopharm. 2000; 15:111; Syrigos K N, et al., Acta Oncol. 1999; 38: 379-382); the MOv18 antibody (Van Zanten-Przbysz J, et al, J Nucl Med. 2000; 41: 1168-1176); the B72.3 antibody (Alvarez R D, et al. Gynecol Oncol. 1997; 65: 94-101); the CC49 antibody (Mulligan T, et al. Clin Cancer Res. 1995; 1: 1447-1454; Macey D J, et al., Clin Cancer Res. 1997; 3: 1547-1555; Meredith R F, et al., Clin Cancer Res. 1999; 5: 3254s-3258s); the MN-14 antibody (Juweid M, et al, Gyn Oncol. 1997; 67: 259-271); the hMN-14 antibody (Behr T M, et al., Clin Cancer Res. 1999; 5: 3232s-3242s; Juweid M E, et al, J Nucl Med. 2000; 41:93-103); the anti-A33 antibody (Heath J K, et al, Proc Natl Acad Sci USA. 1997; 94: 469-474); the NR-LU-10 antibody (Knox S J, et al., Clin Cancer Res. 2000; 6:406-414); HuBrE3 antibody (Cagnoni P J, et al. Cancer Biother Radiophann. 2000; 15:405); the chL6 antibody (Richman C M, et al. Cancer Res. 1995; 55:5916s-5920s); the 170H.82 antibody (Richman C M, et al. Clin Cancer Res. 1999; 5: 3243s-3248s); the J591 antibody (Gong M C, et al., Cancer Metastasis Rev. 1999; 18: 483-490; McDevitt M R, et al., Cancer Res. 2000; 60: 6095-6100); the C595 antibody (Hughes O D, et al, J Clin Oncol. 2000; 18: 363-370) chG250 antibody (Steffens M G, et al., Cancer Res. 1999; 59: 1615-1619); and the F6 antibody (Kraeber-Bodere F, et al., Clin Cancer Res. 1999; 5:3190s-3198s).

In a particularly preferred embodiment, the nanoparticles are targeted to the HER2/neu antigen. HER2/neu is a tumor-associated antigen that has been extensively used as a target for antibody-based detection and treatment of cancer. It is overexpressed on about 25% of breast carcinomas and has been associated with a poor prognosis in recurrent disease (Slamon et al., Science, 1987, 235: 177-182, 1987). It is also overexpressed in adenocarcinomas of the ovary, prostate, lung, and gastrointestinal track (Mehren et al., Annual Reviews of Medicine. 54: 343-369, 2003.), making it an extremely relevant target antigen for radioimmunotherapies. Anti-HER2/neu single chain Fv (scFv) molecules, composed of antibody variable light and variable heavy chains have been isolated from naive human scFv phage display libraries. Affinity mutants have been produced from one such scFv (C6.5) by site-directed mutagenesis and chain shuffling. Affinity constants ranged from 10⁻⁶ to 1.5×10⁻¹¹ M as determined by surface plasmon resonance on a BIAcore instrument (Schier et al., Journal of Molecular Biology, 1996, 263: 551-567; Schier et al., Journal of Molecular Biology, 1996, 255: 28-43, the disclosures of which are incorporated by reference in their entireties and for all purposes).

These scFv molecules have been extensively characterized in biodistribution studies performed in severe combined immunodeficient (scid) mice human with tumor xenografts that overexpress the HER2/neu antigen (e.g., SK—OV-3 tumors) (Adams, et al., Cancer Research, 2001, 61: 4750-4755; Schier et al., Immunotechnology, 1995, 1: 73-81; Adams et al., Cancer Res., 1998, 58: 485-490, the disclosure of which is incorporated by reference in its entirety and for all purposes). They have also been employed in the construction of larger multivalent antibody-based molecules such as diabodies (Adams et al., British Journal of Cancer, 1998, 77: 1405-1412, the disclosures of which are incorporated by reference in their entireties and for all purposes) and minibodies. All of these constructs have been associated with highly specific tumor localization properties, although the highest affinity scFv have exhibited restricted tumor penetration (Adams et al., Cancer Research, 2001, 61: 4750-4755, the disclosure of which is incorporated by reference in its entirety and for all purposes). The C6.5 diabody has also been effectively employed in a series of radioimmunotherapy studies using the beta-emitting radioisotopes yttrium-90 and iodine-131 and the alpha emitting radioisotope astatine-211 to treat established tumors growing in nude mice (Adams et al., Cancer Research, 2001, 61: 4750-4755, the disclosure of which is incorporated by reference in its entireties and for all purposes). The small size (25 kDa) of these anti-HER2/neu scFv molecules make them ideal agents for the creation of nanoparticle radioimmunoconjugates as multiple scFv can be conjugated to a single nanoparticle while still maintaining an overall size that is capable of directing effective tumor targeting. Accordingly the present invention provides radioactive nanoparticles of the present invention conjugated to a ligand specific for the HER2/neu antigen. In particular the present invention provides a radioactive nanoparticle derivatized with, e.g., attached to, a stabilizing material such as, for example, PEG, and an anti-HER-2/neu ScFv molecule such as a C6.5 affinity mutant.

The nanoparticles of the present invention can be associated with ligands specific for endothelial cells. “Endothelial cells” or “endothelium” refers to an aggregate of cells and/or tissue which may be normal and/or diseased and which may comprise a single layer of flattened transparent endothelial cells that may be joined edge to edge or in an overlapping fashion to form a membrane. Endothelial cells are found on the free surfaces of the serous membranes, as part of the lining membrane of the heart, blood vessels, and lymphatics, on the surface of the brain and spinal cord, and in the anterior chamber of the eye. Endothelium originates from the embryonic mesoblast and includes heart tissue, including infarcted heart tissue, cardiovasculature, the peripheral vasculature, such as arteries, veins, and capillaries (the location of which is noted as peripheral to the heart), blood clots and the region surrounding atherosclerotic plaque. Suitable targeting ligands include, for example, growth factors, including, for example, basic fibroblast growth factor (bFGF), acidic fibroblast growth factor (aFGF), transforming growth factor-alpha (TGF-α), transforming growth factor-beta (TGF-β), platelet-derived endothelial cell growth factor (PD-ECGF) vascular endothelial growth factor (VEGF) and human growth factor (HGF); angiogenin; tumor necrosis factors, including tumor necrosis factor-alpha (TNF-α) and tumor necrosis factor-beta (TNF-β). In a particularly preferred embodiment, the nanoparticles are targeted to tumor endothelial markers, for example, TEM1, TEM2, TEM3, TEM4, TEM5, TEM6, TEM7, TEM7R, and TEM8. (Eleanor et al., 2001 Cancer Research 61, 6649-6655, the disclosure of which is incorporated by reference in its entirety and for all purposes).

The nanoparticles of the present invention can be associated with, for example, ligands specific for factors overexpressed during angiogenesis. The development of a vascular supply, commonly referred to as angiogenesis or neovascularization, is essential for the growth, maturation, and maintenance of normal tissues, including neuronal tissues. It is also required for wound healing and the rapid growth of solid tumors and is involved in a variety of other pathological conditions. Current concepts of angiogenesis, based in large part on studies on the vascularization of tumors, suggest that cells secrete angiogenic factors which induce endothelial cell migration, proliferation, and capillary formation. Numerous factors have been identified which induce vessel formation in vitro or in vivo in animal models. These include FGF-α, FGF-β, TGF-α, TNF-α, VPF or VEGF, monobutyrin, angiotropin, angiogenin, hyaluronic acid degradation products, and more recently, B61 for TNF-α induced angiogenesis (Pandey et al., Science, 1995, 268:567-569). The major development of the vascular supply occurs during embryonic development, at ovulation during formation of the corpus luteum, and during wound and fracture healing. Many pathological disease states are characterized by augmented angiogenesis including tumor growth and rheumatoid arthritis. During these processes normally quiescent endothelial cells which line the blood vessels sprout from sites along the vessel, degrade extracellular matrix barriers, proliferate, and migrate to form new vessels. Angiogenic factors, secreted from surrounding tissue, direct the endothelial cells to degrade stromal collagens, undergo directed migration (chemotaxis), proliferate, and reorganize into capillaries. Tumor survival and growth is dependent on angiogenesis, accordingly, in some embodiments of the present invention, it can be desirable to use targeting ligands that target angiogenic factors upregulated during tumor angiogenesis, for example αβ integrins (αᵥβ₃, α₁β₁, α₂β₁ integrins; Cheresh et al., Science 1995, 270, 1500-2; Senger et al., Proc. Natl. Acad. Sci USA, 1997, 94, 13612-7; Reynolds et al., Trends Mol Med., 2003, 9: 2-4., the disclosures of which are incorporated by reference in their entireties and for all purposes). The nanoradiopharmaceuticals of the present invention can be used to treat diseases associated with angiogenesis or to provide images of areas in the body undergoing angiogenesis.

The nanoparticles of the present invention can be conjugated to any ligand capable of selectively targeting a region of interest in a subject. “Region of a patient” refers to a particular area or portion of the patient and in some instances to regions throughout the entire patient. “Region of a patient” includes, for example, regions to be imaged with diagnostic imaging, regions to be treated with a nanoradiopharmaceutical of the present invention, and regions to be targeted for the delivery of a nanoradiopharmaceutical of the present invention. The region of a patient is preferably internal, although it can be external. The phrase “a vasculature region” denotes blood vessels (including arteries, veins and the like). The phrase a “gastrointestinal region” includes the region defined by the esophagus, stomach, small and large intestines, and rectum. The phrase a “renal region” denotes the region defined by the kidney and the vasculature that leads directly to and from the kidney, and includes the abdominal aorta. The phrase “cardiac region” refers generally to the heart and surrounding tissues, structures, and blood vessels, including the coronary arteries. The “region to be targeted” or “targeted region” refer to a region of a patient where delivery of a nanoradioparticle is desired. The “region to be imaged” or an “imaging region” denotes a region of a patient where diagnostic imaging is desired.

The present invention is directed, in part, to radioactive nanoparticles or nanoradioparticle compositions. Embodiments are provided which comprise radioactive nanoparticles or compositions comprising a radioactive nanoparticle in combination with a pharmaceutically acceptable carrier. Embodiments are also provided herein which comprise a radioactive nanoparticle associated with one or more targeting ligand(s) which can target, for example, tissues, cells and/or receptors in vivo or in vitro. Embodiments are also provided which comprise a radioactive nanoparticle associated with one or more targeting ligand(s) and a stabilizing material.

The methods of the present invention provide both in vitro and in vivo applications. Nanoradiopharmaceuticals can be delivered to a subject for diagnostic or therapeutic purposes. The term “subject” or “patient” as used herein means any mammalian patient or subject to which the compositions of the invention can be administered. The term mammals includes human patients and non-human primates, as well as experimental animals such as rabbits, rats, and mice, and other animals, such as dogs and cats. In an exemplary embodiment of the present invention, to identify subject patients for treatment according to the methods of the invention, accepted screening methods are employed to determine risk factors associated with a disease or condition or to determine the status of an existing disease or condition in a subject. These screening methods include, for example, conventional work-ups to determine risk factors that can be associated with the disease or condition. These and other routine methods allow the clinician to select patients in need of therapy using the methods and formulations of the invention.

Nanoradiopharmaceuticals (e.g., nanoradiodiagnostics) of the present invention can be used to irradiate selected biological targets and/or regions including, but not limited to, cells, collection of cells, tissues, organs, and sites of infection. Exemplary of such regions are the pulmonary region, the gastrointestinal region, the cerebellar region, the hippocampal region and other regions of the brain and central nervous system, the cardiovascular region (including myocardial tissue), the renal region as well as other bodily regions, tissues, lymphocytes, receptors, organs and the like, including the vasculature and circulatory system, and as well as diseased tissue, including cancerous tissue.

In some embodiments, the nanoradiopharmaceuticals will be targeted to sites of microbial infection within the body. In such instances, the select biological target can be a foreign species that has invaded the body, e.g., bacterium, virus, parasite or fungus, or a cell infected with a foreign species. Targeting ligands, such as antibodies that bind to antigens on the surface of invading microbes can be associated with the nanoradioparticles and administered to a subject to treat microbial disease, e.g., pneumonia or meningitis. Fungal binding, viral binding and/or bacterial-binding antibodies, e.g., MAb D11, are known in the art and can be used in connection with the present methods (See, e.g., Dadachova et al., The Journal of Nuclear Medicine 2004, 45:2, 313-320; Nosanchuck et al., J Clin Invest. 2003:112:1164-1175, Dadachova et al. Antimicrob. Agents. Chemother. 2004, 48:5, 1624-1629). Exemplary fungal species that can be targeted using the present methods include, but are not limited, to Histoplasma species (including H. capsulatum) and Cryptococcus species (including C. neoformans and C. laurenti). Exemplary bacterial species that can be targed include, but are not limited to, Streptococcus species (including S. pyogenes, S. agalactiae, S. bovis, S. pneumoniae, S. mutans, S. sanguis, S. equi, S. equinus, S. thermophilus, S. morbillorum, S. hansenii, S. pleomorphus, and S. parvulus). Exemplary viruses that can be targed include, but are not limited to, HIV. Microbial agents used in biological warfare such as Bacillus (including B. cereus and B. anthracis) can also be targeted using the methods of the present invention.

The compositions of the present invention can be used as both diagnostic and therapeutic agents. A “diagnostic agent” refers to any substance which can be used in connection with methods for imaging an internal region of a patient and/or diagnosing the presence or absence or progression of a disease or diseased tissue in a patient. A “therapeutic agent,” a “pharmaceutical agent,” or a “drug” refers to any therapeutic or prophylactic agent which can be used in the treatment (including the prevention, diagnosis, alleviation, or cure) of a malady, affliction, condition, disease or injury in a patient.

The term “treating” refers to any indicia of success in the treatment or amelioration of an injury, pathology or condition, including any objective or subjective parameter such as abatement; remission; diminishing of symptoms or making the injury, pathology, or condition more tolerable to the patient; slowing in the rate of degeneration or decline; making the final point of degeneration less debilitating; or improving a subject's physical or mental well-being. The treatment or amelioration of symptoms can be based on objective or subjective parameters; including the results of a physical examination, neurological examination, and/or psychiatric evaluations. The term “treating” includes, for example, the administration of the compounds or agents of the present invention to inhibit tumor angiogenesis, tumor growth, or to cause the regression of already existing tumors. Accordingly, the term “treating” includes, for example, the administration of the compounds or agents of the present invention to prevent or delay, to alleviate, or to arrest or inhibit development of the symptoms or conditions associated with cancers. The term “treating” also includes, for example, the administration of the compounds or agents of the present invention to inhibit microbial infection or kill microbes within a patient. The term “treating” thus also includes, for example, the administration of the compounds or agents of the present invention to alleviate prevent or delay, or to arrest or inhibit development of the symptoms or conditions associated with infection. The term “therapeutic effect” refers to the reduction, elimination, or prevention of the disease, symptoms of the disease, or side effects of the disease in the subject.

Accordingly, any disease or condition treatable by irradiation can be treated using the methods of the present invention. These include proliferative diseases such as cancers and hyperthyroidism. Other diseases include microbial (e.g., pathogenic) infections. Irradiation can also be useful for treating pain, such as pain caused by bone metastasis. The present invention contemplates using the present methods to treat metastatic bone pain.

“Cancer” or “malignancy” are used as synonymous terms and refer to any of a number of diseases that are characterized by uncontrolled, abnormal proliferation of cells, as well as any of a number of characteristic structural and/or molecular features. A “cancerous” or “malignant cell” is understood as a cell having specific structural properties, lacking differentiation and, in many instances, being capable of invasion and metastasis.

“Cancer-associated” refers to the relationship of a nucleic acids and its expression, or lack thereof, or a protein and its level or activity, or lack thereof, to the onset of malignancy in a subject cell. For example, cancer can be associated with expression of a particular gene that is not expressed, or is expressed at a lower level, in a normal healthy cell. Conversely, a cancer-associated gene can be one that is not expressed in a malignant cell (or in a cell undergoing transformation), or is expressed at a lower level in the malignant cell than it is expressed in a normal healthy cell.

As used herein, “neoplastic cells” and “neoplasia” refer to cells which exhibit relatively autonomous growth, so that they exhibit an aberrant growth phenotype characterized by a significant loss of control of cell proliferation. Neoplastic cells comprise cells which can be actively replicating or in a temporary non-replicative resting state (G1 or G0); similarly, neoplastic cells can comprise cells which have a well-differentiated phenotype, a poorly-differentiated phenotype, or a mixture of both type of cells. Thus, not all neoplastic cells are necessarily replicating cells at a given timepoint. The set defined as neoplastic cells consists of cells in benign neoplasms and cells in malignant (or frank) neoplasms. Neoplastic cells are frequently referred to as cancer (discussed supra).

In the context of the cancer, the term “transformation” refers to the change that a normal cell undergoes as it becomes malignant. In eukaryotes, the term “transformation” can be used to describe the conversion of normal cells to malignant cells in cell culture.

“Proliferating cells” are those which are actively undergoing cell division and growing exponentially. “Loss of cell proliferation control” refers to the property of cells that have lost the cell cycle controls that normally ensure appropriate restriction of cell division. Cells that have lost such controls proliferate at a faster than normal rate, without stimulatory signals, and do not respond to inhibitory signals.

Cancers or neoplasms treatable by the methods of the present invention include, but are not limited to, AIDS-Associated cancers; cancers of the femal reproductive organs including, but not limited to, cervical cancer, ovarian cancer, and uterine cancer; lung cancer; renal cell carcinoma; lymphomas (e.g., Hodgkin's or Non-Hodgkin's lymphoma); cancers of the genitourinary system including, but not limited to, stomach cancer, esophageal cancer, small bowel cancer or colon cancer; bladder cancer; bone cancer; brain and spinal cord cancers; metastatic brain tumors; breast cancer; male breast cancer; colorectal cancer; endometrial cancers; gallbladder and bile duct cancers; gestational trophoblastic disease; head and neck cancers; kidney cancer; cancers of the hematopoietic system such as leukemias; liver cancer; liver metastases; melanoma; multiple myelomas; myelodysplastic syndrome; pancreatic cancer; pediatric cancers; pituitary tumors; prostate cancer; rare hematologic disorders; rare solid tumors; skin cancer; soft-tissue sarcoma; testicular cancer; and thyroid cancer. Diseases characterized by excessive angiogenesis, include, but are not limited to, diabetic blindness, age-related macular degeneration, rheumatoid arthritis, and psoriasis. The methods of the present invention can be use to inhibit tumor growth or cause tumor regression. In some embodiments, the tumors will be malignant tumors, e.g., malignant liposarcomas and epithelial tumors. In other embodiments, the tumors will be benign, such as adenomas. Accordingly, disease caused by benign tumors, e.g., acromegaly, are also treatable by the methods of the present invention.

Any disease or condition or abnormal state that can be identified through the use of imaging techniques can be identified using the methods of the present invention. For example, the nanoradioparticles of the present invention can be used in connection with nuclear imaging techniques to image an internal region of a body to determine if the internal region has abnormalities associated with it. Such disease or conditions, include, but are not limited to, those associated with angiogenesis; cancers such as those described above; cardiac diseases, such as atherosclerosis, myocardial infarction, stable and unstable angina, thrombosis; pulmonary diseases, such as; gastrointesteinal disease; renal disease; vasculature diseases; disease of the circulatory system; and neurological disorders.

The compositions of the present invention can also be used in radioimmunoguided surgery (RIGS®) applications, such as gamma knife surgery. For example, a preoperative injection of a gamma-emitting targeted nanoradioparticle of the present invention can be administered to a subject and loci targeted by the nanoradioparticle, e.g., tumors, can be detected with the use of a gamma-detecting probe. Radioimmunoguided surgery can be used to treat a multitude of disease and conditions including, for example, intracranial tumors, such as, for example, acoustic neuromas, pituitary adenomas, pinealomas, craniopharynigiomas, meningiomas, chordomas, chondrosarcomas, metastases and glial tumors; vascular malformations including arteriovenous malformations; functional disorders such as, for example, trigeminal neuralgia, intractable pain, Parkinson's desease and epilepsy.

The compositions of the present invention comprising a gamma ray or positron emitting radionuclide are useful for imaging using known techniques such as gamma scintigraphy or positron emission tomography.

The compositions of the present invention emitting comprising a particle emitting radioactive metal ion, such as β particles, are useful for treating cancer and other proliferative disease by delivering a cytotoxic dose of radiation to the tumors or abnormal cells.

The present invention provides, inter alia, methods of irradiating a selected biological target comprising contacting the target with a nanoradiopharmaceutical of the present invention. The nanoradiopharmaceutical is targeted meaning that it is associated with one or more ligands specific for a biological target of interest. In certain embodiments, the biological target will be associated with a disease state or suspected of being associated with a disease state. In one usual aspect of the present invention, cells, collections of cells, or tissues surrounding the biological target will be irradiated as well. As will be appreciated, the biological target to which nanoradioparticles are associated by virtue of the ligands associated, in turn, with them will be the situs of irradiation. Depending upon the kind of radiation, its effect will be felt at distances more or less remote from the specific site of the particle—target interaction. Thus, it may be seen that irradiation takes place at the locus of the biological target—the vicinity where the target—interaction takes place.

In some preferred embodiments, the selected biological target will be in an internal region of a patient. In such cases, the nanoradiopharmaceuticals of the present invention will be administered to the subject in a pharmaceutically effective amount and in a manner consistent with the formulation, target and other usual parameters. A “pharmaceutically effective amount” refers to the amount that, when administered to a subject is sufficient to have an effect in the subject. For example, when administered for treatment purposes, a “pharmaceutically effective amount” is sufficient to effect treatment. Alternatively, when administered for diagnostic purposes, a “pharmaceutically effective amount” is sufficient to produce an image of an internal region of the subject. For example, in one aspect of the present invention, radiation is provided to the locus of the biological target in an amount sufficient to treat disease, e.g., to cause regression or inhibition of a tumor cell. In such an embodiment, radiation can be provided to the locus of the biological target, for example, in an amount of from about 500 to about 8000 cGy, or from about 1500 to about 7000 cGy, or from about 3000 to about 4500 cGy. In one aspect, radiation is provided to the locus of the biological target at a rate of from about 20 cGy to about 1000 cGy per hour or from about 20 cGy to about 750 cGy per hour or from about 50 cGy to about 500 cGy per hour.

The actual dosage of the nanoradiopharmaceuticals of the present invention will of course vary according to factors such as the extent of disease progression and particular status of the subject (e.g., the subject's age, size, fitness, extent of symptoms, susceptibility factors, etc), time and route of administration, as well as other drugs or treatments being administered concomitantly. Dosage regimens can be adjusted to provide an optimum diagnostic or therapeutic effect. By “therapeutically effective dose” herein is meant a dose that produces effects for which it is administered. The exact dose will be ascertainable by one skilled in the art using known techniques. A therapeutically effective dose is also one in which any toxic or detrimental side effects of the biologically active agent is outweighed in clinical terms by therapeutically beneficial effects. For use in the present invention, an “absorbed dose” refers to the amount of radiation absorbed a particular tissue while an “administered dose” refers to the amount of radiation injected. As used herein, the amount of radiation provided to the locus of the biological target reflects the amount of radiation that will be absorbed by a particular tissue of interest.

Dosage regimens of the pharmaceutical compositions of the present invention are adjusted to provide the optimum desired response (e.g., a therapeutic response). The timing of the administration can vary substantially. The entire dose can be provided in a single bolus. Alternatively, the dose can be provided by an extended infusion method or by repeated injections administered over a span of weeks. For example, in some embodiments, a preferable interval of time is six to twelve weeks between radioimmunotherapeutic doses. Alternatively, the dose can be provided, for example, at two week intervals. In certain embodiments where the total therapeutic dose is fractionally delivered, it can be administered, for example, over a span of 2 to 4 days. In certain embodiments, either or both the diagnostic and therapeutic administrations can be preceded by “pre-doses” of free antibody.

It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular effect to be achieved, and (b) the limitations inherent in the art of compounding such an active compound for the treatment of sensitivity in individuals.

A physician or veterinarian can start doses of the compounds of the invention employed in the pharmaceutical composition at levels lower than that required to achieve the desired therapeutic effect and gradually increase the dosage until the desired effect is achieved. In general, a suitable daily dose of a compound of the invention is that amount of the compound which is the lowest dose effective to produce a therapeutic effect. Such an effective dose generally depends upon the factors described above. It is preferred that administration of the radiopharmaceuticals be intravenous, intramuscular, intraperitoneal, or subcutaneous, or administered proximal to the site of the target.

In one embodiment, the diagnostic radiopharmaceuticals of the present invention can be intravenously injected, preferably in saline solution, at a dose of, for example, from about 0.1 to about 100 mCi, preferably from about 0.1 to about 20 mCi and even more preferably from about 2 to about 10 mCi per 70 kg body weight. Imaging is performed using known procedures. In another embodiment, the radiopharmaceuticals can administered by intravenous injection, preferably in saline solution, at a dose of, for example, from about 0.1 to about 300 mCi, preferably from about 1 to about 300 mCi, more preferably from about 3 to about 200 mCi, and even more preferably from about 10 to about 100 mCi per 70 kg body weight.

The present invention further provides methods of imaging a biological target in a subject or tissue comprising administering to the subject or tissue an imaging agent of the present invention and scanning the subject or tissue to detect the imaging agent. Scanning can include, for example, detecting gamma rays (e.g., by positron emission), detecting electron spin or detecting magnetic resonance.

The present invention further provides methods of selectively destroying tissue in a subject comprising administering to the subject or tissue a nanoradiopharmaceutical of the present invention, scanning the subject to detect the imaging agent, and irradiating a locus of the subject where the imaging agent is detected. In one preferred embodiment, the irradiating step comprises exposing the locus to a plurality of sources of gamma radiation.

The present invention further provides methods of diagnosing the presence or absence of a disease state in a subject or evaluating a disease condition in a subject. Evaluating a disease condition refers to assessing a disease condition of a subject. It can include determining the severity of disease in a subject. It can further include using that determination to make a disease prognosis, e.g., a life-span prediction or treatment plan. Such a method comprises administering to the subject or tissue derived from the subject a radiopharmaceutical of the present invention and detecting the presence or absence of the agent within the subject or tissue. Detection of a certain concentration of particles will be indicative of a disease state.

The present invention further provides methods for inhibiting the development of a cancer in a patient, determining the presence or absence of a cancer in a patient, methods for monitoring the progression of a cancer in a patient and methods of causing tumor regression in a subject comprising administering the nanoradiopharmaceuticals of the present invention.

The therapeutic and diagnostic agents of the present invention can be administered concomitantly with additional diagnostic or therapeutic agents. For example, chemotherapeutic drugs useful in treating neoplastic diseases include alkylating agents, antimetabolites, natural products, hormones and antagonists can be administered concomitantly with the radiopharmaceuticals of the present invention. In another example, antifungal, antiviral, or antibacterial drugs can be administered concomitantly with the nanoradiopharmaceuticals of the present invention. “Concomitant administration” of a known drug with a compound of the present invention means administration of the drug and the compound at such time that both the known drug and the compound will have a therapeutic effect or diagnostic effect. Such concomitant administration can involve concurrent (i.e. at the same time), prior, or subsequent administration of the drug with respect to the administration of a compound of the present invention. A person of ordinary skill in the art, would have no difficulty determining the appropriate timing, sequence and dosages of administration for particular drugs and compounds of the present invention.

The present invention provides pharmaceutical compositions comprising a pharmaceutically effective amount of the nanoradiopharmaceuticals described herein and a physiologically acceptable carrier or diluent. Pharmaceutical compositions of the present invention can further comprise pharmaceutically acceptable excipients. As used herein, the term “carrier” refers to a pharmaceutically-acceptable carrier, adjuvant or vehicle that can be administered to a patient, together with the diagnostic and therapeutic agents of this invention, and which does not destroy the activity thereof and is nontoxic when administered in doses sufficient to deliver an effective amount of the diagnostic or therapeutic agent. The carrier can be a solvent or dispersion medium containing, for example, water, ethanol, polyol (for example, glycerol, propylene glycol, polyethylenes such as polytheylene glycol, and the like), and suitable mixtures thereof. The carrier can be suitable for intravenous, intramuscular, subcutaneous, parenteral, spinal or epidermal administration (e.g., by injection or infusion).

The pharmaceutical compositions of the present invention can comprise pharmaceutically acceptable excipients meaning excipients that are useful in preparing a pharmaceutical composition that is generally safe and desirable, and includes excipients that are acceptable for veterinary use as well as for human pharmaceutical use. Such excipients can be solid, liquid, or semisolid. They can include, for example, buffers, lyophilization aids, stabilization aids, solubilization and anti-agglomeration aids and bacteriostats.

Buffers useful in the preparation of the radiopharmaceuticals of the present invention can include, but are not limited to, phosphate, citrate, sulfosalicylate, and acetate. A more complete list can be found in the United States Pharmacopeia, the disclosure of which is incorporated by reference in its entirety and for all purposes.

Lyophilization aids can include, but are not limited to, mannitol, lactose, sorbitol, sodium chloride, dextran, Ficoll, polyvyinylpyrrolidine, and the like.

Stabilization aids can include, but are not limited to, ascorbic acid, cysteine, monothioglycerol, sodium bisulfite, sodium metabisulfite, gentisic acid, inositol, and the like.

Solubilization aids can include, but are not limited to ethanol, glycerin, polyethylene glycol, polysorbates, polyoxyethylene sorbitan monooleate, sorbitan monooleate, Pluronic copolymers, ethylene vinyl acetate, polyanhydrides, polyglycolic acid, collagen, polyorthoesters, polylactic acid and the like.

Bacteriostats can include, but are not limited to, benzyl alcohol, benzalkonium chloride, chlorbutanol, and the like.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects which would be to a degree that would prohibit administration of the composition. The pharmaceutical compositions are generally formulated in full compliance with all Good Manufacturing Practice (GMP) regulations of the U.S. Food and Drug Administration.

The present invention also provides kits for diagnostic imaging and the therapeutic administration of radioactive nanoparticles. In one embodiment, the kit comprises a pharmaceutical composition comprising a nanoradiopharmaceutical and instructions for the administration of the nanoradiopharmaceutical. In another embodiment, the kit comprises a reducing agent, buffer salt, targeting ligand and/or stabilizing ligand such as a capping agent, and instructions for the preparation of the nanoradiopharmaceuticals of the present invention.

Although the foregoing invention has been described in detail by way of example for purposes of clarity of understanding, it will be apparent to the artisan that certain changes and modifications are comprehended by the disclosure and can be practiced without undue experimentation within the scope of the appended claims, which are presented by way of illustration not limitation.

All publications and patent documents cited above are hereby incorporated by reference in their entirety for all purposes to the same extent as if each were so individually denoted.

### EXAMPLES

### Example 1

### Exemplary Synthesis Procedure for Rhenium-Rich Nanoparticles

Rhenium-rich nanoparticles were synthesized using the following reagents: reaction buffer (200 mM acetate, pH 4.5), elution buffer (20 mM sodium ascorbate, pH 8.2), 100 mM NaReO₄ in ultrapure water, dihydrolipoic acid (DHLA), sodium borohydride, and 500 mM sodium bicarbonate.

450 μL water, 1000 μL reaction buffer, and 50 μL NaReO₄ solution were mixed in a test tube and stirred while purging with dry nitrogen for 30 min at room temperature. While the reactants were being stirred, DHLA was dissolved in bicarbonate solution at 10 mg/mL. (DHLA solution was pH 8-9.) A solution of NaBH₄ in ultrapure water was prepared at 7.57 mg/mL (200 mM). After the 30 min elapsed, 500 μL NaBH₄ solution was added to the test tube, dropwise over 60 seconds. The solution turned from clear to tan and was ˜pH 7 after this step. 60 seconds after completion of NaBH₄ addition, 1000 μL DHLA solution was added to the test tube. The solution was ˜pH 8 after this step. The solution was stirred with nitrogen purge for 30 min at room temperature. A PD-10 desalting column (5000 NMWL) was equilibrated with 25 mL elution buffer. After the 30 min elapsed, 2.5 mL of solution was applied to the column and entered the gel. The flow-through was discarded. 3.5 mL of elution buffer was applied to the column. The flow-through, which contained the nanoparticles, was collected. The flow-through was stirred with nitrogen purge for 30 min. The flow-through was filtered through a 220 μm PVDF membrane for size analysis by light scattering. Yield was ˜50 nmol 2-3 nm diameter metal core nanoparticles.

### Example 2

### Scanning Transmission Electron Microscopy (STEM)

The morphology and size of the nanoparticles synthesized by the method described in example 1 were determined by STEM. The sample was prepared after elution through the PD-10 column by pipetting a 5 μL drop of nanoparticle solution onto an electron microscopy grid coated with a carbon support film. The drop was allowed to sit for 3 minutes before it was wicked away with filter paper. Some of the nanoparticles remained on the carbon support film. The particles were visualized and measured to be approximately 2-3 nm in diameter.

The X-rays emitted from the sample during STEM imaging were detected with an energy dispersive spectrometer. A set of characteristics X-rays corresponding to rhenium were detected, confirming the composition of the nanoparticles.

### Example 3

### Dynamic Light Scattering

The size of the nanoparticles synthesized by the method described in example 1 was measured by light scattering. After filtration through the PVDF membrane, the size distribution of the nanoparticles in the sample was determined. Mean hydrodynamic particle size was measured to be approximately 4-5 nm, which is consistent with a 2-3 nm diameter core surrounded by DHLA ligands and a counterion layer.

### Example 4

### Exemplary Procedure for Preparation of Targeted Rhenium-Rich Nanoparticles

Targeted rhenium-rich nanoparticles are synthesized using the following reagents: reaction buffer (200 mM acetate, pH 4.5), elution buffer (20 mM sodium ascorbate, pH 8.2), 100 mM NaReO₄ in ultrapure water, dihydrolipoic acid (DHLA), sodium borohydride, and 500 mM sodium bicarbonate. Biomolecules with targeting capability are also utilized. The biomolecules are thiolated in advance (e.g. with 2-iminothiolane or by carbodiimide coupling to DHLA).

450 μL water, 1000 μL reaction buffer, and 50 μL NaReO₄ solution are mixed in a test tube and stirred while purging with dry nitrogen for 30 min at room temperature. While the reactants are being stirred, DHLA and thiolated biomolecules are dissolved in bicarbonate solution. A solution of NaBH₄ in ultrapure water is prepared at 7.57 mg/mL (200 mM). After the 30 min elapsed, 500 μL NaBH₄ solution is added to the test tube, dropwise over 60 seconds. The solution turns from clear to tan and is ˜pH 7 after this step. 60 seconds after completion of NaBH₄ addition, the thiolated biomolecules are added with DHLA to the test tube. The solution is stirred with nitrogen purge for 30 min at room temperature. A PD-10 desalting column (5000 NMWL) is equilibrated with 25 mL elution buffer. After the 30 min elapses, 2.5 mL of solution is applied to the column and enters the gel. The flow-through is discarded. 3.5 mL of elution buffer is applied to the column. The flow-through, which contains the biomolecule-functionalized nanoparticles, is collected. The flow-through is stirred with nitrogen purge for 30 min. The flow-through is filtered through a 220 μm PVDF membrane.

### Example 5

### Exemplary Procedure for the Preparation of Rhenium-Rich Nanoparticles

The following synthesis reactions were preferably performed at room temperature in a test tube held in a bath sonicator. Continuous sonication helps to prevent agglomeration of the unprotected nanoparticles during nucleation and growth.

Rhenium-rich nanoparticles were synthesized using the following reagents: 0.1 M ascorbic acid, 0.1 M sodium ascorbate, 50 mM NaReO₄, 0.1 M 2-mercaptoacetic acid (2 MA), 0.3 N HNO3, and sodium borohydride. All solutions are prepared in ultrapure water.

Water, ascorbic acid, sodium ascorbate, and NaReO₄ solutions were mixed in a test tube to give 2.5 μmol NaReO₄ in 875 μL of 68.5 mM ascorbate buffer, pH 4.1. A 0.3 M solution of NaBH₄ in ultrapure water was prepared. 125 μL NaBH₄ solution (37.5 μmol) was added to the test tube, corresponding to 1:15 mole ratio for NaReO₄:NaBH₄. The reaction solution turned from clear to tan. The reaction solution was sonicated for 60 minutes. 125 μL HNO3 solution was added to the test tube. After an additional 60 seconds, 250 μL 2 MA solution was added to the test tube.

Nanoparticles produced by the method described in example 5 were visualized with transmission electron microscopy. The particles were measured to range from 1-3 nm in diameter.

1. A method for preparing a nanoradiopharmaceutical comprising:
a. reducing a radionuclide-containing moiety in aqueous medium with a reducing agent under conditions selected to form particles having a mean diameter of from 1 to about 25 nanometers; and
b. associating with the particles ligand specific for a biological target, the average number of radioactive atoms per particle is at least about five.

2. The method of claim 1 wherein the ligand is associated with the particles in said aqueous medium.

3. The method of claim 1 wherein the particles have a mean diameter of from about 2 to about 5 nanometers.

4. The method of claim 1 wherein the nanoradiopharmaceutical is synthesized in less than about 5 hours.

5. The method of claim 1 wherein the radionuclide moiety is an oxy-anion.

6. The method of claim 1 wherein the reducing agent is a metal hydride.

7. The method of claim 1 wherein the radionuclide comprises Rhenium 186, Rhenium 188, Copper 64, Copper 67, Gold 198, Gold 199, Silver 111, Rhodium 105, Palladium 109, Iridium 194, Technetium 99m, Technetium 94 or mixtures thereof.

8. The method of claim 7 wherein the radionuclide is Rhenium 186, Rhenium 188, or mixtures thereof.

9. The method of claim 1 wherein ligand is attached to at least one of the particles.

10. The method of claim 1 wherein the ligand comprises a plurality of individual ligands attached to at least some of the particles.

11. The method of claim 10 wherein the ligand is an immunologically active moiety.

12. The method of claim 11 wherein the ligand is an antibody.

13. The method of claim 1 wherein the specificity of the ligand does not depend upon immunological interactions.

14. The method of claim 1 further comprising the step of associating the particles with a stabilizing moiety.

15. The method of claim 14 wherein the stabilizing moiety is a hydrophilic polymer.

16. The method of claim 1 wherein the biological target is a tissue, organ, cell, or collection of cells.

17. The method of claim 1 wherein the ligand binds specifically to a cell receptor.

18. The method of claim 17 wherein the cell receptor is an antigen.

19. The method of claim 17 wherein the cell receptor is implicated in a disease state.

20. The method of claim 19 wherein the disease state is a neoplastic disease.

21. The method of claim 19 wherein the disease state is a microbial infection.

22. The method of claim 1 wherein the ligand binds specifically to a cell surface marker.

23. The method of claim 22 wherein the cell surface marker is implicated in a disease state.

24. The method of claim 22 wherein the marker is not, itself, implicated in a disease state.

25. The method of claim 1 further comprising a step of placing a surface coating on at least one of said particles.

26. The method of claim 25, wherein said coating is an inorganic, metal or oxide surface coating.

27. A method for preparing a nanoradiopharmaceutical comprising:
a. reducing a radionuclide-containing moiety in aqueous medium with a reducing agent under conditions selected to form particles having a mean diameter of from 1 to about 25 nanometers; and
b. in said medium, associating with the particles ligand specific for a biological target.

28. The method of claim 27 wherein the average number of radioactive atoms per particle is at least about five.

29. The method of claim 27 wherein the nanoradiopharmaceutical is synthesized in less than about 5 hours.

30. The method of claim 27 further comprising the step of associating the particles with a stabilizing moiety.

31. The method of claim 27 wherein the biological target is a tissue, organ, cell, or collection of cells.

32. The method of claim 27 wherein the ligand binds specifically to a cell receptor.

33. The method of claim 32 wherein the cell receptor is an antigen.

34. The method of claim 33 wherein the cell receptor is implicated in a disease state.

35. The method of claim 34 wherein the disease state is a neoplastic disease.

36. The method of claim 34 wherein the disease state is a microbial infection.

37. The method of claim 27 further comprising a step of placing a surface coating on at least one of said particles.

38. The method of claim 37, wherein said surface coating is an inorganic, metal or oxide surface coating.

39. A nanoradiopharmaceutical comprising an aqueous dispersion of nanoparticles derived from the reduction of a radionuclide precursor moiety by a reducing agent in aqueous medium and the association of ligand specific for a biological target in said medium;
a. the particles having a mean diameter of from 1 to about 25 nanometers;
b. the particles having a ligand associated therewith, the ligand being specific for a biological target, wherein the average number of radioactive atoms per particle is at least about five.

40. A method for irradiating a selected biological target comprising contacting the target with a nanoradiopharmaceutical;
a. the nanoradiopharmaceutical comprising an aqueous dispersion of nanoparticles comprising a radionuclide;
b. the particles having a mean diameter of from 1 to about 25 nanometers;
c. the particles having a ligand associated therewith, the ligand being specific for the biological target, wherein the average number of radioactive atoms per particle is at least about five.

41. A method for irradiating a selected biological target in a subject comprising administering to the subject a nanoradiopharmaceutical;
a. the nanoradiopharmaceutical comprising an aqueous dispersion of nanoparticles comprising a radionuclide;
b. the particles having a mean diameter of from 1 to about 25 nanometers;
c. the particles having a ligand associated therewith, the ligand being specific for the biological target, wherein the average number of radioactive atoms per particle is at least about five.

42. The method of claim 41 wherein radiation is provided to the locus of the biological target in an amount of from about 500 to about 5000 cGY.

43. The method of claim 41 wherein the biological target is a tissue, organ, cell, or collection of cells.

44. The method of claim 41 wherein the biological target is a pathogen.

45. A method of imaging a biological target in a subject or tissue comprising administering to the subject or tissue an imaging agent comprising an aqueous dispersion of nanoparticles derived from the reduction of a radionuclide precursor moiety by a reducing agent in aqueous medium;
a. the particles having a mean diameter of from 1 to about 25 nanometers;
b. the particles having a ligand associated therewith, the ligand being specific for the biological target, wherein the average number of radioactive atoms per particle is at least about five; and
scanning the subject or tissue to detect the imaging agent

46. The method of claim 45 wherein the scanning detects positrons.

47. The method of claim 46 wherein the scanning detects gamma rays.

48. The method of claim 45 wherein the scanning detects electron spin.

49. The method of claim 45 wherein the scanning is magnetic resonance.

50. A method of selectively destroying tissue in a subject comprising
a. imaging a biological target in the subject, the imaging comprising administering to the subject an imaging agent comprising an aqueous dispersion of nanoparticles derived from the reduction of a radionuclide precursor moiety by a reducing agent in aqueous medium;
i. the particles having a mean diameter of from 1 to about 25 nanometers;
ii. the particles having a ligand associated therewith, wherein the ligand was associated with the particles in said aqueous medium, the ligand being specific for a biological target associated with the tissue to be selectively destroyed wherein the average number of radioactive atoms per particle is at least about five;
b. scanning the subject to detect the imaging agent; and
c. irradiating a locus of the subject where the imaging agent is detected.

51. The method of claim 50 wherein said irradiating comprising exposing the locus to a plurality of sources of gamma radiation.

52. A method of diagnosing the presence of a disease state in a patient or tissue comprising administering to the subject or tissue an imaging agent comprising an aqueous dispersion of nanoparticles derived from the reduction of a radionuclide precursor moiety by a reducing agent in aqueous medium;
a. the particles having a mean diameter of from 1 to about 25 nanometers;
b. the particles having a ligand associated therewith, wherein the ligand was associated with the particles in said aqueous medium, the ligand being specific for a biological target implicated in the disease state and wherein the average number of radioactive atoms per particle is at least about five; and
c. detecting the presence of the nanoparticles within the subject or tissue; a concentration of particles implying the presence of the disease state.

53. A method for treating a disease or condition associated with a known or suspected biological target in an organism comprising administering to a subject the nanoradiopharmaceutical of claim 39.

54. A pharmaceutical composition comprising a therapeutically effective amount of the nanoradiopharmaceutical of claim 39 and a pharmaceutically acceptable carrier or diluent.

55. An imaging composition comprising a therapeutically effective amount of the nanoradiopharmaceutical composition of claim 39 and a physiologically acceptable carrier or diluent.

56. A kit for the preparation of a nanoradiopharmaceutical comprising a reducing agent, a buffering agent, a targeting ligand and instructions for performing the method of claim 1.
